(19) United States
(12) Patent Application Publication
(10) Pub. No.: US 2025/0321162 A1
(43) Pub. Date: Oct. 16, 2025

(54) METHOD AND DEVICE FOR ANALYSING A DEVICE FOR SPRAYING A PHARMACEUTICAL FLUID PRODUCT

(71) Applicant: APTAR FRANCE SAS, LE NEUBOURG (FR)

(72) Inventor: Guillaume GRANJON, LA VACHERIE (FR)

(73) Assignee: APTAR FRANCE SAS, LE NEUBOURG (FR)

(21) Appl. No.: 18/866,315

(22) PCT Filed: May 15, 2023
(86) PCT No.: PCT/FR2023/050690

§ 371 Date: Nov. 15, 2024

(30) Foreign Application Priority Data
May 16, 2022 (FR) FR2204608

(51) Int. Cl. G01M 99/00; A61M 11/00

(52) U.S. Cl. CPC G01M 99/008; A61M 11/00; A61M 2205/0272; A61M 2205/3306; A61M 2205/70

(57) ABSTRACT

A method for analysing a device for spraying a pharmaceutical fluid product, the method including the steps of providing a spraying head including a spray opening; providing a receiving zone forming a container the bottom of which is formed by a plate behind which a camera is arranged, the container containing a ferrofluid; providing, under the plate, a set of magnets generating a magnetic charge to force the ferrofluid to cover the plate; passing a stream of gas through the spraying opening, and sending it over the receiving zone, the ferrofluid that is hit by the stream of gas being moved over the plate to form an impact zone; viewing the impact zone with the camera; and analysing the view of the impact zone in order to determine whether or not the impact zone complies with predetermined specifications.

The present invention relates to a device and to a method for analysing a spray generated by a device for spraying a pharmaceutical fluid product.

Spray devices for spraying pharmaceutical fluid product are well known. They generally comprise a spraying head provided with a spraying opening, assembled on a container containing the fluid product to be distributed. Particularly in nasal spray applications, the therapeutic effectiveness of the sprayed fluid product may depend on the properties of the spray generated while the device is being actuated. At the end of the assembly line, i.e. once the spray device has been assembled and just prior to being sent to the pharmaceutical fluid product manufacturer for assembly there onto a corresponding container, it is known for a certain number of samples of assembled devices to be laboratory tested in order to check whether the properties of the spray correspond to pre-defined production specifications.

A disadvantage with that system is that it pertains to assembled devices, and thus destroys those devices which, after having been tested, can no longer be delivered to the customer.

Furthermore, the system requires human verification of the tested devices, and is thus not suitable for being completely automated.

To overcome this disadvantage, the document WO 2018/130791 proposes visualization of a stream of hot or cold compressed air sent through a spraying head by strioscopy. That method makes it possible to evaluate the angle of the spray, but not its geometry, nor its symmetry. This also has the disadvantage of having to provide a strioscopic bench, which is relatively complex and expensive, and which is difficult to adapt to an assembly line for a fluid spray device, and therefore involves either random tests carried out on only a portion of the manufactured devices, or slowing down the assembly line, which is generally undesirable.

Document EP3047912 describes a visualization system and method using the detection of a temperature difference between a stream of heated gas passing through a spraying head and a receiving zone at ambient temperature, to determine the compliance of said spraying head. This solution requires heating means and the use of a thermal camera, which makes the assembly complex and expensive to use.

Document WO2022003294 also describes a visualization system and method using the detection of a temperature difference with thermosensitive detection means.

Documents JPH0599802 and JPS54127347 describe other devices of the prior art.

An object of the present invention is to overcome the above-mentioned drawbacks.

In particular, the aim of the present invention is to provide a device and a method for analysing a device for spraying a pharmaceutical fluid product that do not involve the destruction of the tested devices.

The present invention also aims to provide a device and a method for analysing which is substantially automated.

The present invention also aims to provide a device and method for analysing which makes it possible to test 100% of the spraying devices, without slowing down the assembly line to a substantial extent.

The present invention also aims to provide a device and method for analysing which uses neither heating means, nor thermal camera, nor thermosensitive detection means.

Another aim of the present invention is to provide a device and method for analysing which is simple and/or inexpensive to manufacture, assemble and use.

What is therefore presented is a method for analysing a device for spraying a pharmaceutical fluid product, comprising the following steps:
providing a spraying head for a device for spraying a pharmaceutical fluid product, said spraying head comprising a spraying opening;
providing a receiving zone forming a container the bottom of which is formed by a transparent plate behind which a camera is arranged, said container containing a ferrofluid,
providing, under said transparent plate, a set of magnets generating a magnetic charge to force said ferrofluid to cover said transparent plate,
passing a stream of gas through said spraying opening of said spraying head, and sending it over said receiving zone, said ferrofluid contacted by said stream of gas being moved over said transparent plate to form an impact zone,
visualising said impact zone with said camera, and
with means for analysing, analysing said visualisation of said impact zone in order to determine whether or not said impact zone complies with predetermined specifications.

Advantageously, said stream of gas is a stream of compressed gas.

Advantageously, said stream of compressed gas is a compressed air stream.

Advantageously, said step for analysing comprises determining the geometry, in particular the symmetry, of the impact zone for said stream of gas over said receiving zone.

Advantageously, said predetermined specifications comprise a predetermined planar extent of the impact zone for said stream of gas over said receiving zone, in a manner such that the spraying heads for which said planar extent is similar to said predetermined planar extent are classified as compliant, and the spraying heads for which said planar extent is different from said predetermined planar extent are classified as non-compliant.

Advantageously, the operating cycle comprises the following steps:
activating said set of magnets to generate a magnetic charge to force said ferrofluid to cover said transparent plate,
generating said stream of gas and sending it through said spraying head over said receiving zone, the ferrofluid contacted by said stream of gas being pushed back towards the edges of said receiving zone to form an impact zone over said transparent plate,
viewing said impact zone by taking an image of the balls on said pane by means of said camera.

The present invention also concerns a device for analysing a device for spraying a pharmaceutical fluid product, comprising:
a spraying head for a device for spraying a pharmaceutical fluid product, said spraying head comprising a spraying opening;
a receiving zone forming a container the bottom of which is formed by a transparent plate, said container containing a ferrofluid,
a set of magnets generating a magnetic charge to force said ferrofluid to cover said transparent plate,
means for generating a stream of gas in order to pass a stream of gas through said spraying opening of said spraying head and sending it over said receiving zone, the ferrofluid contacted by said stream of gas being moved over said transparent plate to form an impact zone,
a camera arranged under said transparent plate for viewing said impact zone for said stream of compressed gas over said transparent plate by taking an image of said ferrofluid moved by said stream of gas over said transparent plate, and
means for analysing (40), for analysing said image of said impact zone in order to determine whether or not said impact zone complies with predetermined specifications.

Advantageously, said stream of compressed gas is a stream of compressed air.

Advantageously, said ferrofluid is a colloidal suspension of ferromagnetic or ferrimagnetic nanoparticles in a solvent or water.

Advantageously, said means for generating a stream of compressed gas are adapted to generate pulses of adjustable duration, in particular from 50 to 300 ms.

Advantageously, said magnet assembly comprises magnets and/or electromagnets.

These characteristics and advantages and others appear more clearly from the following detailed description, given by way of non-limiting example, and with reference to the accompanying drawings, in which:

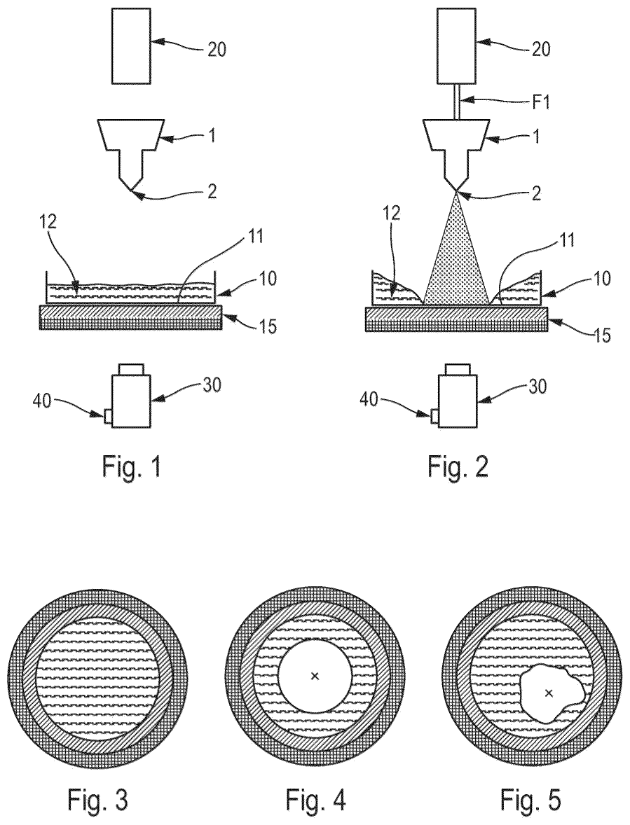

FIG. 1 is a diagrammatic view of a device for analysing a spray device in accordance with an advantageous embodiment, before actuation,

FIG. 2 is a view similar to the view in FIG. 1, during actuation,

FIG. 3 shows a visualisation of the receiving zone at rest,

FIG. 4 shows a visualisation of a compliant impact zone, and

FIG. 5 shows a visualisation of a non-compliant impact zone.

One aim of the invention is to improve the quality of spray device inspection. To this end, the invention envisages the analysis of 100% of the devices, without substantially slowing down the assembly line.

In conventional manner, each device for spraying comprises a spraying head 1 provided with a spraying opening 2. In general, a spraying profile (not shown) is provided upstream of said spraying opening 2 in order to generate a conical spray shape at the outlet from the opening.

The present invention envisages passing a stream of gas F1, preferably compressed, through each spraying head 1, and directing this stream of compressed gas F1 leaving the spraying opening 2 in the form of a conical spray towards a receiving zone 10. Advantageously, the stream of gas F1 is a compressed air stream, but it should be understood that, in accordance with the invention, any suitable gas other than air could be used.

FIGS. 1 and 2 shows a test device according to an advantageous embodiment.

In this example, a spraying head 1 is disposed opposite a receiving zone 10. Means 20 for generating a stream of compressed gas F1 are provided in order to cause a stream of compressed gas F1 to pass through the spraying head 1.

The receiving zone 10 forms a container the bottom of which is formed by a transparent plate 11. This container contains a ferrofluid 12. A set of magnets 15 generating a magnetic charge is arranged under the transparent plate 11 to force the ferrofluid 12 to cover said transparent plate 11. Advantageously, the magnet assembly 15 forms a ring defining an empty central portion through which the transparent plate 11 remains visible. The magnet assembly 15 may include magnets and/or electromagnets.

Ferrofluids are colloidal suspensions of ferromagnetic or ferrimagnetic nanoparticles, typically about 10 nanometers in size, in a solvent or water. These liquids become magnetic during the application of an external magnetic field while maintaining their colloidal stability. Ferrofluids are most commonly composed of nanoparticles of magnetite (Fe3O4) or maghemite (γ-Fe2O3), both of which are iron oxides.

When the stream of gas F1 is sent over the receiving zone 10, the ferrofluid 12 deforms under the effect of the stream of gas and is therefore pushed outwards, to concentrate at the walls of the container. What is not blown by the stream of gas F1 is held in place by the magnetic charge.

Thus, the part of the transparent plate 11 devoid of ferrofluid 12 after sending the stream of gas F1 corresponds to the impact zone of said stream of gas F1 over the receiving zone 10.

A camera 30 is arranged behind the transparent plate 11, in order to visualise this impact zone.

Thus, the operation of the device of the example of FIGS. 1 and 2 is as follows.

The magnet assembly 15 generates a magnetic charge that forces the ferrofluid 12 to substantially homogeneously cover the transparent plate 11 of the container forming the receiving zone 10, as can be seen in FIG. 1.

A stream of gas F1, in particular of compressed air, is then generated and sent through the spraying head 1 towards the receiving zone 10. The force of the stream of gas F1 deforms the ferrofluid 12 over the perforated plate 11 by surmounting the magnetic charge of the magnet assembly 15. In other words, the part of the ferrofluid 12 that is hit by said stream of gas F1 will be pushed towards the edges of the plate 11, as illustrated in FIG. 2.

The camera 30 then takes an image of this plate 11, with the ferrofluid 12 having been hit by the stream of gas F1 forming the impact zone on said plate 11.

After each use, as soon as the stream of gas F1 stops, the ferrofluid 12 returns under the effect of the magnetic charge to its initial position in which it covers the plate 11, and it is possible to restart a new test almost immediately.

In order to carry out the compliance evaluations, a camera 30 is provided to view the impact zone and means for analysing 40 are provided to analyse the views generated by the camera 30 and thus determine whether the impact zone of the stream of gas F1 coming from said spraying head 1 over the receiving zone 10 is compliant or not with predetermined specifications.

The duration of the gas pulse F1 is advantageously adjustable, in particular from 50 to 300 ms.

Advantageously, it is possible to carry out a plurality of successive cycles on the same spraying head, for example five cycles. The consistency or repeatability of the results also makes it possible to evaluate the compliance of said spraying head.

The predetermined specifications may comprise a predetermined planar extent of said impact zone on said receiving zone 10, in a manner such that the spray heads 1 for which said planar extent is similar to said predetermined planar extent are classified as compliant, and the spray heads 1 for which said planar extent is different from said predetermined planar extent are classified as non-compliant. The geometry, in and particular the symmetry, of the impact zone may also be used in the compliance evaluation. Other parameters may also be envisaged.

The means for analysing 40 may comprise means for measuring the geometry of the impact zone of the stream of gas F1 over the receiving zone 10. As an example, the centre of mass of the impact zone is determined, and the maximum and minimum distances of this centre of mass from the edge of the impact zone are measured. Comparing these distances with predetermined values then makes it possible to evaluate the compliance of the tested device. Thus, the compliance evaluation takes not only the surface of the impact zone into account, but also its geometry, in particular its symmetry. This makes it possible to establish that a spray leaving a compliant spraying head will have an acceptable conical shape, both from the point of view of the angle of the spray and as regards its symmetry.

Optionally, image processing means may be used to carry out this type of analysis.

FIGS. 4 and 5 each illustrate a diagrammatic representation obtained with the method and the device of the invention, in which it is possible to evaluate the planar extent and the geometry, in particular the symmetry, of the impact zone. FIG. 4 shows a view of the impact zone for a compliant device and FIG. 5 shows such a view for a non-compliant device.

The present invention presents numerous advantages, and in particular:
it enables various types of device for spraying to be inspected in an automated manner;
it enables said devices for spraying to be analysed non-destructively;
it makes it possible to analyse 100% of the devices for spraying assembled on an assembly line, without slowing down the assembly line substantially;
it makes it possible to carry out several successive tests on the same device in order to evaluate the repeatability of the results;
it uses a setup that is compact and that can easily be adapted;
it uses components which are simple and standard, and thus generally inexpensive;
it requires neither heating means, nor thermal camera, nor thermosensitive detection means;
it enables image processing to be robust, and it can be carried out in real time;
it guarantees good repeatability and good discrimination between compliant and non-compliant devices.

The present invention has been described with reference to an advantageous embodiment, but naturally any modification could be applied thereto by the person skilled in the art, without going beyond the scope of the present invention, as defined by the accompanying claims.

1. A method for analysing a device for spraying a pharmaceutical fluid product, characterised in that it comprises the following steps:
providing a spraying head for a device for spraying a pharmaceutical fluid product, said spraying head comprising a spraying opening,
providing a receiving zone forming a container the bottom of which is formed by a transparent plate behind which a camera is arranged, said container containing a ferrofluid,
providing, under said transparent plate, a set of magnets generating a magnetic charge to force said ferrofluid to cover said transparent plate,
passing a stream of gas through said spraying opening of said spraying head, and sending it over said receiving zone, said ferrofluid contacted by said stream of gas being moved over said transparent plate to form an impact zone,
viewing said impact zone with said camera, and
means for analysing, for analysis said view of said impact zone in order to determine whether or not said impact zone complies with predetermined specifications.

2. The method as claimed in claim 1, wherein said stream of gas is a stream of compressed gas.

3. The method as claimed in claim 1, wherein said stream of gas is a compressed air stream.

4. The method as claimed in claim 1, wherein said step for analysing comprises determining the geometry, in particular the symmetry, of the impact zone for said stream of gas over said receiving zone.

5. The method as claimed in claim 1, wherein said predetermined specifications comprise a predetermined planar extent of the impact zone for said stream of gas over said receiving zone, in a manner such that the spraying heads for which said planar extent is similar to said predetermined planar extent are classified as compliant, and the spraying heads for which said planar extent is different from said predetermined planar extent are classified as non-compliant.

6. The method as claimed in claim 1, wherein an operating cycle comprises the following steps:
activating said set of magnets to generate a magnetic charge to force said ferrofluid to cover said transparent plate,
generating said stream of gas and sending it through said spraying head over said receiving zone, the ferrofluid contacted by said stream of gas being pushed back towards the edges of said receiving zone to form an impact zone over said transparent plate,
viewing said impact zone by taking an image of said transparent plate by means of said camera.

7. A device for analysing a device for spraying a pharmaceutical fluid product, characterised in that it comprises:
a spraying head of a device for spraying a pharmaceutical fluid product, said spraying head comprising a spraying opening;
a receiving zone forming a container the bottom of which is formed by a transparent plate, said container containing a ferrofluid,
a set of magnets generating a magnetic charge to force said ferrofluid to cover said transparent plate,
means for generating a stream of gas in order to pass a stream of gas through said spraying opening of said spraying head and sending it over said receiving zone, said ferrofluid contacted by said stream of gas being moved over said transparent plate to form an impact zone,
a camera arranged under said transparent plate for viewing said impact zone of said stream of compressed gas over said transparent plate by taking an image of said ferrofluid moved by said stream of gas over said transparent plate, and
means for analysing, for analysing said image of said impact zone in order to determine whether or not said impact zone complies with predetermined specifications.

8. The device as claimed in claim 7, wherein said stream of gas is a compressed air stream.

9. The device as claimed in claim 7, wherein said ferrofluid is a colloidal suspension of ferromagnetic or ferrimagnetic nanoparticles in a solvent or water.

10. The device as claimed in claim 7, wherein said means for generating a stream of compressed gas are adapted to generate pulses of adjustable duration, in particular from 50 to 300 ms.

11. The device as claimed in claim 7, wherein said magnet assembly comprises magnets and/or electromagnets.
